(19) United States
(12) Patent Application Publication
(10) Pub. No.: US 2006/0039981 A1
(43) Pub. Date: Feb. 23, 2006

(54) Taste masked dosage forms and processes for their preparation

(76) Inventors: Deepak Murpani, New Delhi, Delhi (IN); Vinod Kumar Arora, Delhi (IN); Rajiv Malik, Vienna (AU)

Correspondence Address: RANBAXY INC., 600 COLLEGE ROAD EAST, SUITE 2100, PRINCETON, NJ, 08540, US

(21) Appl. No.: 10/526,844

(22) PCT Filed: Sep. 4, 2003
(86) PCT No.: PCT/IB03/03779

§ 371 Date: Jul. 27, 2005

(30) Foreign Application Priority Data
Sep. 4, 2002 (IN) 903/DEL/2002

(51) Int. Cl. A61K 9/14

(52) U.S. Cl. 424/487

(57) ABSTRACT

The invention relates to taste masked dosage forms utilizing low amounts of taste masking polymer, and simple and economical processes for the preparation of the taste masked dosage forms. The taste-masked dosage form includes one or more drugs and one or more cationic polymers synthesized from dimethylaminoethyl methacrylate and neutral methacrylic acid esters. The wt/wt ratio of the drug to polymer is less than about one to two.

### FIELD OF THE INVENTION

The technical field of the invention relates to taste masked dosage forms utilizing low amounts of taste masking polymer, and simple and economical processes for the preparation of such taste masked dosage forms.

### BACKGROUND OF THE INVENTION

Many patients, especially children and elderly, have trouble in swallowing whole tablets and even capsules. It is therefore desirable to administer drugs to such patients either as a liquid dosage form or as a fast dissolving or fast disintegrating solid dosage form. Fast dissolving or disintegrating solid dosage forms, due to their ease of administration and pleasant taste, may encourage patients to adhere to daily medication regimens and therefore provide better compliance. These dosage forms combine the advantages of both liquid and conventional tablet formulations, and also offer advantage over both traditional dosage forms. For example, they provide the convenience of a tablet formulation while also allowing the ease of swallowing provided by a liquid formulation. They also allow the luxury of much more accurate dosing than the primary alternative, oral liquids.

Palatability and “mouth feel” are among the most important characteristics to be considered in providing fast dissolving or disintegrating solid dosage forms, or matrix, for a drug. Unfortunately, many drugs have a bitter or otherwise unpalatable taste, or an unacceptable mouth feel, which make such drugs unsuitable for administration as fast dissolving or fast disintegrating dosage forms. Much research has been devoted to designing techniques and approaches to mask the bitter taste of drug in dosage forms. Simple approaches include adding chemicals mediating, flavoring or sweetening ingredients to the composition, which thereby mask the bitterness of the drug. When simple approaches are ineffective, drug modifying approaches are used in which the dosage form is so formulated that the drug's dissolution in the mouth is retarded or prevented by physical and/or chemical means. One such approach to retard by physical means is to embed or encapsulate the drug within a wall or barrier material that physically separates it from the saliva. Cationic copolymers synthesized from dimethylaminoethyl methacrylate and neutral methacrylic acid have been employed as the barrier material in various taste-masked formulations. In some cases, these polymers are also known to modify taste by chemically interacting with drugs.

For instance, U.S. Pat. No. 5,286,489 discloses a method of preparing taste masked dosage forms of active ingredients having an amine or amido groups by making a porous drug-polymer matrix with Eudragit® E-100. U.S. Pat. No. 5,275,823 discloses a chewable tablet that includes a granulate of a histamine H2-receptor antagonist and Eudragit E® 100, and an admixture of a taste-masking extragranular water-insoluble hygroscopic excipient. U.S. Pat. No. 5,489,436 discloses a chewable medicament tablet that includes a medicament coated with a taste-masking amount of a polymer blend of dimethylaminoethyl methacrylate and neutral methacrylic acid esters and a polymer selected from cellulose acetate and cellulose triacetate. U.S. Pat. No. 4,708,867 discloses a mini pellet dosage form of prednisone. The dosage form includes a nonpareil seed coated with a first layer of the drug and a second layer of a copolymer of dimethylaminoethyl methacrylate and methyl methacrylate. U.S. Pat. No. 4,760,093 discloses a taste neutral powder form of spray-dried acetaminophen which includes about 60% to 74% by weight acetaminophen and about 26% to 40% by weight of a copolymer that is cationic in character and is based on dimethylaminoethyl methacrylate and neutral methacrylic acid esters.

U.S. Pat. No. 6,153,220 discloses use of cationic copolymers synthesized from dimethylaminoethyl methacrylate and neutral methacrylic acid esters in amounts significantly greater than the amount of drug in need of taste masking to form with the drug a taste masked micromatrix powder. The drug and the copolymer (e.g., Eudragit® E 100) are in the form of micromatrices having an average size from about 1 μm to 125 μm. The '220 patent states that the ratio of copolymer to drug is greater than two to one and that the prior art does not teach the advantageous use of employing cationic copolymers synthesized from dimethylaminoethyl methacrylate and neutral methacrylic acid esters in amounts significantly greater than the amount of drug in need of taste masking to form with the drug a taste-masked micromatrix powder.

The processes used for taste masking in the patents listed above involve multiple steps which are technically complicated and difficult to reproduce, besides being economically disadvantageous. Moreover the recommended limit by FDA for oral intake of polymer with a dimethylaminoethyl group is quite low and therefore these polymers in practice cannot be used in higher amounts. Therefore, there still exists a need for taste masked dosage forms utilizing low amounts of cationic polymers.

### SUMMARY OF THE INVENTION

In one general aspect there is provided a taste-masked pharmaceutical dosage form that includes one or more drugs and one or more cationic polymers synthesized from dimethylaminoethyl methacrylate and neutral methacrylic acid esters. The wt/wt ratio of the drug to polymer is less than about one to two.

Embodiments of the dosage form may include one or more of the following features. For example, the wt/wt ratio of the drug to polymer may be less than approximately 1:1.7 or less than approximately 1:1.5.

The drug may be one or more of H₂ receptor antagonists, antibiotics, analgesics, cardiovascular agents, peptides or proteins, hormones, anti-migraine agents, anti-coagulant agents, anti-emetic agents, anti-hypertensive agents, narcotic antagonists, chelating agents, anti-anginal agents, chemotherapeutic agents, sedatives, anti-neoplastics, prostaglandins, drugs for erectile dysfunction, drugs acting on central nervous system, anti-diarrhoeal and anti-diuretic agents. The drug may be one or more of nizatidine, cimetidine, ranitidine, famotidine, roxatidine, etinidine, lupitidine, nifentidine, niperitone, sulfotidine, tuvatidine, zaltidine, erythomycin, penicillin, ampicillin, roxithromycin, clarithromycin, psylium, ciprofloxacin, theophylline, nifedipine, prednisone, prednisolone, ketoprofen, acetaminophen, ibuprofen, dexibuprofen lysinate, flurbiprofen, naproxen, codeine, morphine, sodium diclofenac, acetylsalicylic acid, caffeine, pseudoephedrine, phenylpropanolamine, diphenhydramine, chlorpheniramine, dextromethorphan, berberine, mefenamic acid, flufenamic acid, astemizole, terfenadine, phenytoin, guiafenesin, N-acetylprocainamide HCl and pharmaceutically acceptable salts or derivatives thereof.

The drug may be one more unpleasant tasting drugs. The drug may be a low dose drug and the low dose drug may be one or more of enalapril, lorazepam, zolmitriptan, domperidon, selegiline, ondansetron, mirtazepine, hyosyamine sulphate, risperidone, citalopram, olanzapine, rizatriptan, piroxicam, desloratadine, cetirizine, loperamide, sildenafil, topiramate, and pharmaceutically acceptable salts or derivatives thereof.

The cationic polymer may include a dimethylaminoethyl group. The cationic polymer may have the following formula: 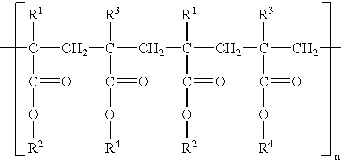
where: R¹═R³═CH₃
R²═CH₂CH₂N(CH₃)₂
R⁴═CH₃, C₄H₉.
   The cationic polymer may be a polymers commercially available as Eudragit®. The Eudragit® may be one or both of Eudragit® E-100 and Eudragit® EPO.

The taste masked pharmaceutical dosage form may further include other additives. The additives may be one or more f cellulose ester, talc, magnesium stearate and pigments. The cellulose ester may be one or more of cellulose acetate, cellulose acetate butyrate, cellulose triacetate, ethyl cellulose and mixtures thereof.

A drug solution/dispersion may be coated onto a water soluble or insoluble inert core. The water soluble or insoluble inert core may include one or more of directly compressible dibasic calcium phosphate, directly compressible sugar, microcrystalline cellulose, and nonpareil sugar seeds. The inert core may be directly compressible mannitol. The inert core may have a particle size greater than about 100 microns.

The dosage form may be one or more of sprinkles, chewable tablets, mouth dissolving tablets, water dispersible tablets, effervescent tablets and suspensions. The dosage form may further include one or more pharmaceutically inert excipients. The one or more pharmaceutically inert excipient may be one or more of diluents, binders, disintegrants, coloring agents, flavoring agents, stabilizers, surfactants, lubricants, glidants, plasticizers and preservatives.

In another general aspect there is provided a process for the preparation of a taste masked dosage form of one or more unpleasant tasting drugs. The process includes dissolving or dispersing one or more drugs and one or more cationic polymers in a solvent; and loading a solution and/or dispersion of one or more drugs and one or more cationic polymer onto an inert core. The wt/wt ratio of the drug to polymer in the dosage form is less than about one to two. The one or more cationic polymers are synthesized from dimethylaminoethyl methacrylate and neutral methacrylic acid esters

Embodiments of the process may include one or more of the features described above or the following features. For example, the loading of the drug solution/dispersion over the inert core may be carried out by one or more of granulation, spray coating or coacervation technique. The solvent may include one or more of acetone, methanol, ethyl alcohol, isopropyl alcohol, water, n-butyl alcohol, propylene glycol, ethylene glycol, monobutyl ether, methyl ethyl ketone, cyclohexanone, methylene chloride, chloroform, carbon tetrachloride, trichloroethylene, tetrachloroethylene, ethyl acetate, n-butyl acetate, propylene glycol acetate, toluene and mixtures thereof. The cationic polymer may include a dimethylaminoethyl group. The cationic polymer may have the following formula: 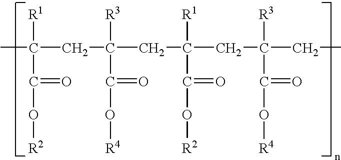
where: R¹═R³═CH₃
R²═CH₂CH₂N(CH₃)₂
R⁴═CH₃, C₄H₉.
   The cationic polymer may include a polymer commercially available as Eudragit®. The Eudragit® may be one or both of Eudragit® E-100 and Eudragit® EPO.

In another general aspect there is provided a taste masked pharmaceutical dosage form that includes an inert core; one or more drugs; and one or more cationic polymers. The one or more cationic polymers are synthesized from dimethylaminoethyl methacrylate and neutral methacrylic acid esters, the one or more drugs and the one or more cationic polymers form a layer around the inert core, and the wt/wt ratio of the drug to polymer in the dosage form is less than about one to two.

Embodiments of the dosage form may include one or more of the features described above or the following features. For example, the cationic polymer may include a dimethylaminoethyl group. The cationic polymer may have the following formula: 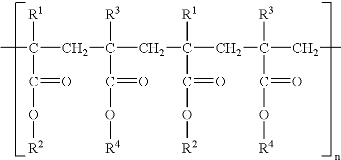
where: R¹═R³═CH₃
R²═CH₂CH₂N(CH₃)₂
R⁴═CH₃, C₄H₉.
   The cationic polymer may be a polymer commercially available as Eudragit®. The Eudragit® may be one or both of Eudragit® E-100 and Eudragit® EPO.

The inert core may be one or more of directly compressible dibasic calcium phosphate, directly compressible sugar, microcrystalline cellulose, and nonpareil sugar seeds.

The details of one or more embodiments of the inventions are set forth in the description below. Other features, objects, and advantages of the inventions will be apparent from the description and the claims.

### DETAILED DESCRIPTION OF THE INVENTION

The present invention involves a single step process for the preparation of a taste masked dosage form which requires low amounts of cationic polymer. Hence, there is provided a taste masked dosage form comprising unpleasant tasting drug and low amount of cationic polymer. The cationic polymer may have a dimethylaminoethyl group. In another general aspect there is provided a process for the preparation of the taste masked dosage form of unpleasant tasting drug wherein the process includes loading of a solution/dispersion of the drug and the low amount of cationic polymer on to an inert core. Again, the cationic polymer may have a dimethylaminoethyl group. In particular, the weight ratio of the amounts of drug and cationic polymer in the dosage form is less than about one to two.

The taste masked dosage forms are prepared by dispersing and/or dissolving one or more drugs and one or more cationic polymers in a solvent and loading this solution or dispersion onto cores. Unlike other processes in which a separated drug coat and polymer coat is used in a multi-step process, the taste masked dosage forms are formed in a single step process. Moreover, the quantity of the polymer required to mask the unpleasant taste of the drug is reduced relative to the prior art multi-step processes, which is not only economical, but also provides better maneuverability for other excipients. Further, it provides a physical polymeric barrier, which completely embeds and/or surrounds the drug particles unlike in other coating processes in which the particle shape or deposition in a dead zone may not allow complete particle coating. Further, as the drug and polymer get mixed intimately, it prevents breaking of taste masking coating by mastication. Moreover complete solubility of the cationic polymer with a dimethylaminoethyl group in acidic pH assures complete drug dissolution in the upper gastro intestinal tract.

These drug-loaded cores may be further processed into dosage forms such as sprinkles, chewable tablets, mouth dissolving tablets, water dispersible tablets, effervescent tablets and suspensions.

Examples of the therapeutic categories of drugs suitable for the taste masked dosage form include H₂ receptor antagonists, antibiotics, analgesics, cardiovascular agents, peptides or proteins, hormones, anti-migraine agents, anti-coagulant agents, anti-emetic agents, anti-hypertensive agents, narcotic antagonists, chelating agents, anti-anginal agents, chemotherapy agents, sedatives, anti-neoplastics, prostaglandins, drugs for erectile dysfunction, drugs acting on central nervous system, anti-diarrhoeal antidiuretic agents, and generally any other drug for which taste masking is desired.

Specific examples of drugs of the above therapeutic categories include but are not limited to nizatidine, cimetidine, ranitidine, famotidine, roxatidine, etinidine, lupitidine, nifentidine, niperitone, sulfotidine, tuvatidine, zaltidine, erythomycin, penicillin, ampicillin, roxithromycin, clarithromycin, psylium, ciprofloxacin, theophylline, nifedipine, prednisone, prednisolone, ketoprofen, acetaminophen, ibuprofen, dexibuprofen lysinate, flurbiprofen, naproxen, codeine, morphine, sodium diclofenac, acetylsalicylic acid, caffeine, pseudoephedrine, phenylpropanolamine, diphenhydramine, chlorpheniramine, dextromethorphan, berberine, mefenamic acid, flufenamic acid, astemizole, terfenadine, phenytoin, guiafenesin, N-acetylprocainamide hydrochloride, and pharmaceutically acceptable salts or derivatives thereof.

In particular, low dose drugs such as enalapril, lorazepam, zolmitriptan, domperidon, selegiline, ondansetron, mirtazepine, hyosyamine sulphate, risperidone, citalopram, olanzapine, rizatriptan, piroxicam, desloratadine, cetirizine, loperamide, sildenafil, and topiramate and pharmaceutically acceptable salts or derivatives thereof may be used.

Examples of cationic polymers with dimethylaminoethyl groups include various grades of polymers commercially available from Rohm Pharma, Germany. In particular, Eudragit® E-100 and Eudragit® EPO may be used. In presence of an acid, Eudragit® E-100 and Eudragit® EPO form water soluble salts thus providing gastrosoluble film coatings. Eudragit® E films swell and are permeable in water and buffer solutions above pH 5 and is soluble in gastric fluid below pH 5. The average molecular weight of Eudragit® E is about 150,000 and it neither contains any plasticizers nor requires their addition for processing. The Eudragit® E-100 is present in an amount sufficient to mask the otherwise disagreeable taste of the medicament while in the mouth of the user. The drug to Eudragit® ratio generally is less than or equal to one to two and, in particular is about 1:1.75.

Eudragit®E polymers are methacrylic acid derivatives with a dimethylaminoethyl group. According to the fourth addition of the Handbook of Pharmaceutical Excipients, Eudragit E is a cationic polymer based on dimethylaminoethyl methacrylate and other neutral methacrylic acid esters. It is soluble in gastric fluid as well as in weakly acidic buffer solutions (up to pH of approximately 5). The structure of Eudragit E is given in the handbook as: 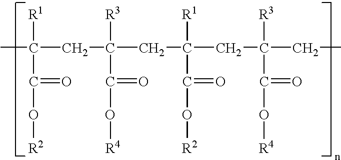
where: R¹═R³═CH₃
R²═CH₂CH₂N(CH₃)₂
R⁴═CH₃, C₄H₉

In one of the embodiments, the taste masked dosage form may further include other additives such as cellulose esters, talc, magnesium stearate and pigments which decrease the tendency of the Eudragit® polymer to agglomerate and thereby produce a more uniform surface on the nonpareil seed. Appropriate examples of cellulose esters include cellulose acetate, cellulose acetate butyrate, cellulose triacetate, ethyl cellulose and mixtures thereof.

Examples of suitable inert cores include water soluble and water insoluble particles, ideally having a size greater than about 100 microns. Specific examples of suitable seeds or cores that may be used in the dosage forms include inert cores prepared from directly compressible dibasic calcium phosphate; directly compressible sugar such as directly compressible mannitol commercially available as PEARLITOL® SD 200 by Roquette Freres S. A., France; microcrystalline cellulose such as those commercially available as Ethispheres®, made of 100% microcrystalline cellulose and which offers a good alternative for sugar-sensitive users and are available in particle sizes of 200 to 1000 micron; and nonpareil sugar seeds marketed by different manufacturers under different trade names. These are available in different sizes ranging from 20 to 2000 microns.

Besides the above materials, the taste masked dosage form may include one or more pharmaceutically inert excipients such as diluents, binders, disintegrants, coloring agents, flavoring agents, stabilizers, surfactants, lubricants/glidants, plasticizers and preservatives which are well known in the art of pharmaceutical formulations.

In another embodiment, taste masked dosage forms of unpleasant tasting drugs may be prepared by preparing a solution and/or dispersion of one or more unpleasant tasting drug and a low amount of one or more cationic polymers, optionally with other additives and loading the inert core with the above solution/dispersion of drug; and forming into a suitable dosage form. Again, the one or more cationic polymers may have a dimethylaminoethyl ammonium group

The solution/dispersion of the drug may be loaded over the inert core using any conventional technique known in the prior art such as granulation, spray coating, or coacervation techniques. In particular, the spray coating technique may be used.

Loading of the solution/dispersion of the drug over the inert core by a spray coating technique may be carried out by a process that includes the steps of dissolving the unpleasant tasting drug and cationic polymer in the solvent and spraying the solution over inert cores in a fluidized bed coater, such as Glatt Fluid Bed Wurster HS Coater. Air is passed through a bed of the inert core particles to fluidize them, and the solvent solution of the drug-polymer is sprayed onto the fluidized bed. The air passing through the bed dries the loaded core particles. The drug loaded cores may then be used in combination with various excipients, flavors, and colors to make a chewable, water dispersible or mouth dissolving tablet. These drug loaded cores may also be placed in a capsule to provide sprinkle capsules or may be suspended in suitable solvent to make suspensions.

Loading by a granulation process may be carried out by conventional techniques using a rapid mixer granulator or a fluid bed granulator. For loading by a coacervation process, homogenizer may be used.

Examples of suitable organic solvents used for the preparation of the solution/dispersion of drug include acetone, methanol, ethyl alcohol, isopropyl alcohol, water and mixtures thereof. Other examples include n-butyl alcohol, propylene glycol, ethylene glycol, monobutyl ether, methyl ethyl ketone, cyclohexanone, methylene chloride, chloroform, carbon tetrachloride, trichloroethylene, tetrachloroethylene, ethyl acetate, n-butyl acetate, propylene glycol acetate, toluene and mixtures thereof.

The following examples further exemplify the inventions and are not intended to limit the scope of the inventions

### EXAMPLE 1

| Ingredient | Quantity (mg) |
|---|---|
| Topiramate | 15 |
| Eudragit ® EPO | 26 |
| Ethyl cellulose (low viscosity) | 3.7 |
| Titanium dioxide | 1.0 |
| Nonpareil seeds | 45.3 |
| Talc | 8.9 |
| Isopropyl alcohol/Water (3:1) | q.s. |
| Total | 100 |

Process:

Weighed quantities of topiramate, Eudragit® EPO and ethyl cellulose were dissolved in a suitable quantity of an isopropyl alcohol/water mixture to prepare the drug-polymer solution. Talc and titanium dioxide were then added to the above solution. Nonpareil seeds were placed in a Glatt Fluid Bed Wurster HS Coater and a drug polymer solution was sprayed on them. The resulting coated beads were cured by keeping them at room temperature for 24 hours. These coated beads were filled into a hard gelatin capsule. The formulation of Example 1 had a ratio of drug (topirimate) to cationic polymer (Eudragit® EPO) of 15 to 26 (i.e., 1 to 1.733).

### EXAMPLE 2

| Ingredient | Quantity (mg) |
|---|---|
| Desloratadine | 5.05 |
| Eudragit ® E PO | 7.50 |
| Ethyl cellulose | 5.0 |
| Talc | 5.0 |
| Isopropyl alcohol | q.s. |
| Water | q.s. |
| Nonpareil seeds | 20.0 |
| Total | 42.55 |

Process:

Weighed quantities of desloratadine, Eudragit® EPO and ethyl cellulose were dissolved in a suitable quantity of an isopropyl alcohol/water mixture to prepare the drug-polymer solution. Talc was then added to the above solution. Nonpareil seeds were placed in a Glatt Fluid Bed Wurster HS Coater and the drug-polymer solution was sprayed on them. The resulting coated beads were cured by keeping them at room temperature for 24 hours. These coated beads were filled into a hard gelatin capsule. The formulation of Example 2 had a ratio of drug (desloratadine) to cationic polymer (Eudragit® EPO) of 5.05 to 7.50 (i.e., 1 to 1.49).

### EXAMPLE 3

| Ingredient | Quantity (gm) |
|---|---|
| Desloratadine | 20.2 |
| Eudragit ® E PO | 30.0 |
| Ethyl cellulose | 20.0 |
| Talc | 20.0 |
| Isopropyl alcohol | q.s. |
| Water | q.s. |
| Nonpareil seeds | 80.0 |
| Total | 170.20 |

Process:

The process for producing the formulation of Example 3 was the same as the process used for Example 2. The formulation of Example 3 had a ratio of drug (desloratadine) to cationic polymer (Eudragit® EPO) of 20.2 to 30.0 (i.e., 1 to 1.49).

While several particular forms of the inventions have been described, it will be apparent that various modifications and combinations of the inventions detailed in the text can be made without departing from the spirit and scope of the inventions. Finally, it is contemplated that any single feature or any combination of optional features of the inventive variations described herein may be specifically excluded from the claimed inventions and be so described as a negative limitation. Accordingly, it is not intended that the inventions be limited, except as by the appended claims.

We claim:

1. A taste-masked pharmaceutical dosage form comprising one or more drugs and one or more cationic polymers synthesized from dimethylaminoethyl methacrylate and neutral methacrylic acid esters, wherein the wt/wt ratio of the drug to polymer is less than about one to two.

2. The taste masked pharmaceutical dosage form according to claim 1 wherein the wt/wt ratio of the drug to polymer is less than approximately 1:1.7.

3. The taste masked pharmaceutical dosage form according to claim 1 wherein the wt/wt ratio of the drug to polymer is less than approximately 1:1.5.

4. The taste masked pharmaceutical dosage form according to claim 1 wherein the drug comprises one or more of H₂ receptor antagonists, antibiotics, analgesics, cardiovascular agents, peptides or proteins, hormones, anti-migraine agents, anti-coagulant agents, anti-emetic agents, anti-hypertensive agents, narcotic antagonists, chelating agents, anti-anginal agents, chemotherapeutic agents, sedatives, anti-neoplastics, prostaglandins, drugs for erectile dysfunction, drugs acting on central nervous system, anti-diarrhoeal and anti-diuretic agents.

5. The taste masked pharmaceutical dosage form according to claim 1 wherein the drug comprises one or more of nizatidine, cimetidine, ranitidine, famotidine, roxatidine, etinidine, lupitidine, nifentidine, niperitone, sulfotidine, tuvatidine, zaltidine, erythomycin, penicillin, ampicillin, roxithromycin, clarithromycin, psylium, ciprofloxacin, theophylline, nifedipine, prednisone, prednisolone, ketoprofen, acetaminophen, ibuprofen, dexibuprofen lysinate, flurbiprofen, naproxen, codeine, morphine, sodium diclofenac, acetylsalicylic acid, caffeine, pseudoephedrine, phenylpropanolamine, diphenhydramine, chlorpheniramine, dextromethorphan, berberine, mefenamic acid, flufenamic acid, astemizole, terfenadine, phenytoin, guiafenesin, N-acetylprocainamide HCl and pharmaceutically acceptable salts or derivatives thereof.

6. The taste masked pharmaceutical dosage form according to claim 1 wherein the drug comprises one more unpleasant tasting drugs.

7. The taste masked pharmaceutical dosage form according to claim 1 wherein the drug comprises a low dose drug.

8. The taste masked pharmaceutical dosage form according to claim 7 wherein the low dose drug comprises one or more of enalapril, lorazepam, zolmitriptan, domperidon, selegiline, ondansetron, mirtazepine, hyosyamine sulphate, risperidone, citalopram, olanzapine, rizatriptan, piroxicam, desloratadine, cetirizine, loperamide, sildenafil, topiramate, and pharmaceutically acceptable salts or derivatives thereof.

9. The taste masked pharmaceutical dosage form according to claim 1 wherein the cationic polymer includes a dimethylaminoethyl group.

10. The taste masked pharmaceutical dosage form according to claim 1 wherein the cationic polymer has the following formula: 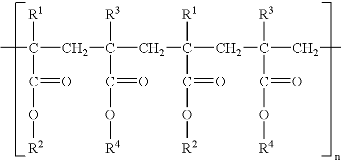
where: R¹═R³═CH₃
R²═CH₂CH₂N(CH₃)₂
R⁴═CH₃, C₄H₉.

11. The taste masked pharmaceutical dosage form according to claim 1 wherein the cationic polymer comprises a polymers commercially available as Eudragit®.

12. The taste masked pharmaceutical dosage form according to claim 11 wherein the Eudragit® comprises one or both of Eudragit® E-100 and Eudragit® EPO.

13. The taste masked pharmaceutical dosage form according to claim 12 wherein the Eudragit® comprises Eudragit® E-100.

14. The taste masked pharmaceutical dosage form according to claim 12 wherein the Eudragit® comprises Eudragit® EPO.

15. The taste masked pharmaceutical dosage form according to claim 1 wherein the dosage form further comprises other additives.

16. The taste masked pharmaceutical dosage form according to claim 15 wherein the additives comprise one or more f cellulose ester, talc, magnesium stearate and pigments.

17. The taste masked pharmaceutical dosage form according to claim 16 wherein the cellulose ester comprises one or more of cellulose acetate, cellulose acetate butyrate, cellulose triacetate, ethyl cellulose and mixtures thereof.

18. The taste masked pharmaceutical dosage form according to claim 1 wherein a drug solution/dispersion is coated onto a water soluble or insoluble inert core.

19. The taste masked pharmaceutical dosage form according to claim 18 wherein the water soluble or insoluble inert core comprises one or more of directly compressible dibasic calcium phosphate, directly compressible sugar, microcrystalline cellulose, and nonpareil sugar seeds.

20. The taste masked pharmaceutical dosage form according to claim 19 wherein the inert core comprises directly compressible mannitol.

21. The taste masked pharmaceutical dosage form according to claim 18 wherein the inert core has a particle size greater than about 100 microns.

22. The taste masked pharmaceutical dosage form according to claim 1 wherein the dosage form is selected from the group consisting of sprinkles, chewable tablets, mouth dissolving tablets, water dispersible tablets, effervescent tablets and suspensions.

23. The taste masked pharmaceutical dosage form according to claim 1 wherein the dosage form further comprises one or more pharmaceutically inert excipients.

24. The taste masked pharmaceutical dosage form according to claim 23 wherein the one or more pharmaceutically inert excipient comprise one or more of diluents, binders, disintegrants, coloring agents, flavoring agents, stabilizers, surfactants, lubricants, glidants, plasticizers and preservatives.

25. A process for the preparation of a taste masked dosage form of one or more unpleasant tasting drugs, the process comprising:
dissolving or dispersing one or more drugs and one or more cationic polymers in a solvent; and
loading a solution and/or dispersion of one or more drugs and one or more cationic polymer onto an inert core
wherein the one or more cationic polymers are synthesized from dimethylaminoethyl methacrylate and neutral methacrylic acid esters and the wt/wt ratio of the drug to polymer in the dosage form is less than about one to two.

26. The process according to claim 25 wherein the loading of the drug solution/dispersion over the inert core is carried out by one or more of granulation, spray coating or coacervation technique.

27. The process according to claim 25 wherein the loading of the drug solution/dispersion over the inert core is carried out by spray coating.

28. The process according to claim 25 wherein the loading of the drug solution/dispersion over the inert core is carried out by granulation.

29. The process according to claim 25 wherein the loading of the drug solution/dispersion over the inert core is carried out by coacervation.

30. The process according to claim 25 wherein the solvent comprises one or more of acetone, methanol, ethyl alcohol, isopropyl alcohol, water, n-butyl alcohol, propylene glycol, ethylene glycol, monobutyl ether, methyl ethyl ketone, cyclohexanone, methylene chloride, chloroform, carbon tetrachloride, trichloroethylene, tetrachloroethylene, ethyl acetate, n-butyl acetate, propylene glycol acetate, toluene and mixtures thereof.

31. The process according to claim 25 wherein the cationic polymer includes a dimethylaminoethyl group.

32. The process according to claim 25 wherein the cationic polymer has the following formula: 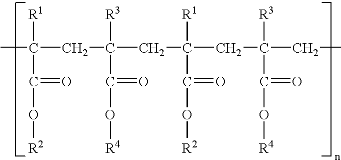
where: R¹═R³═CH₃
R²═CH₂CH₂N(CH₃)₂
R⁴═CH₃, C₄H₉.

33. The process according to claim 25 wherein the cationic polymer comprises a polymer commercially available as Eudragit®.

34. The process according to claim 25 wherein the Eudragit® comprises one or both of Eudragit® E-100 and Eudragit® EPO.

35. A taste masked pharmaceutical dosage form comprising:
an inert core;
one or more drugs; and
one or more cationic polymers,
wherein one or more cationic polymers are synthesized from dimethylaminoethyl methacrylate and neutral methacrylic acid esters, the one or more drugs and the one or more cationic polymers form a layer around the inert core, and the wt/wt ratio of the drug to polymer in the dosage form is less than about one to two.

36. The taste masked pharmaceutical dosage form according to claim 35 wherein the cationic polymer includes a dimethylaminoethyl group.

37. The taste masked pharmaceutical dosage form according to claim 35 wherein the cationic polymer has the following formula: 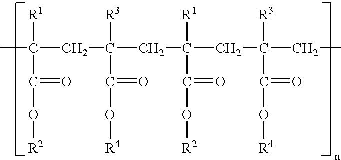
where: R¹═R³═CH₃
R²═CH₂CH₂N(CH₃)₂
R⁴═CH₃, C₄H₉.

38. The taste masked pharmaceutical dosage form according to claim 35 wherein the cationic polymer comprises a polymer commercially available as Eudragit®.

39. The taste masked pharmaceutical dosage form according to claim 35 wherein the Eudragit® comprises one or both of Eudragit® E-100 and Eudragit® EPO.

40. The taste masked pharmaceutical dosage form according to claim 35 wherein the inert core comprises one or more of directly compressible dibasic calcium phosphate, directly compressible sugar, microcrystalline cellulose, and nonpareil sugar seeds.
